(19) United States
(12) Patent Application Publication
(10) Pub. No.: US 2023/0404683 A1
(43) Pub. Date: Dec. 21, 2023

(54) Systems and Methods for Automatically Recommending a Medical Device for Vascular Access

(71) Applicant: Bard Access Systems, Inc., Salt Lake City, UT (US)

(72) Inventors: Jeffrey H. Schmidt, Salt Lake City, UT (US); Steffan Sowards, Salt Lake City, UT (US); William Robert McLaughlin, Bountiful, UT (US); Anthony K. Misener, Bountiful, UT (US)

(21) Appl. No.: 17/841,541

(22) Filed: Jun. 15, 2022

(51) Int. Cl. A61B 34/00; A61B 8/08; G16H 10/60

(52) U.S. Cl. CPC A61B 34/25; A61B 8/085; A61B 8/5292; G16H 10/60; A61B 2034/256; A61B 2034/107

(57) ABSTRACT

Disclosed are systems and methods for automatically recommending medical devices for vascular access. For example, a system can include an ultrasound probe, a console operably coupled to the ultrasound probe, and a display screen integrated into the console. The console can include one or more processors and memory including instructions configured to instantiate one or more processes when executed by the one-or-more processors for the automatic recommending of the medical device for vascular access. The automatic recommending of the medical device for vascular access can be in accordance with a plurality of data inputs, various operating parameters, or a combination thereof. The display screen can be configured to display an ultrasound image including one or more blood vessels below a skin surface of a patient. The display screen can also be configured to display the medical device recommended for vascular access of the one-or-more blood vessels in the ultrasound image.

### BACKGROUND

Current technologies work to optimize placement of a user-selected medical device for vascular access; however, the user-selected medical device is not always the best medical device for vascular access in view of multifaceted considerations of applicable criteria.

Disclosed herein are systems and methods for automatically recommending medical devices for vascular access to ensure best procedural outcomes starting with medical-device selection.

### SUMMARY

Disclosed herein is a system for automatically recommending a medical device for vascular access. The system includes, in some embodiments, an ultrasound probe, a console operably coupled to the ultrasound probe, and a display screen optionally integrated into the console. The console includes one or more processors and memory including instructions configured to instantiate one or more processes when executed by the one-or-more processors for the automatic recommending of the medical device for vascular access. The automatic recommending of the medical device for vascular access is in accordance with a plurality of data inputs, various operating parameters, or a combination thereof using at least logic, algorithms, machine learning, artificial intelligence, or a combination thereof. The display screen is configured to display an ultrasound image including one or more blood vessels below a skin surface of a patient. The display screen is also configured to display the medical device recommended for vascular access of the one-or-more blood vessels in the ultrasound image.

In some embodiments, the plurality of data inputs are automatically determined by the system by way of the logic, the algorithms, the machine learning, the artificial intelligence, or the combination thereof.

In some embodiments, the plurality of data inputs are selected from blood-vessel size, blood-vessel location, blood-vessel cross section, blood-vessel thickness, and difficulty in blood-vessel access.

In some embodiments, the blood-vessel location includes a determination of a distance from a lens of the ultrasound probe to a top of a blood vessel, a bottom of the blood vessel, or both.

In some embodiments, a largest distance between the lens of the ultrasound probe and the bottom of the blood vessel is a determination parameter.

In some embodiments, the difficulty in blood-vessel access includes a determination of whether a blood vessel is visible, palpable, torturous, valvular, or a combination thereof.

In some embodiments, the logic, the algorithms, the machine learning, the artificial intelligence, or the combination thereof are further for automatically guiding optimum blood-vessel size, differentiating between veins and arteries, or both.

In some embodiments, the logic, the algorithms, the machine learning, the artificial intelligence, or the combination thereof are further for automatically detecting a needle guide and presetting an angle of insertion for the needle guide.

In some embodiments, the automatic recommending of the medical device for vascular access is further in accordance with a plurality of patient condition parameters input by an input device. The plurality of patient condition parameters include temperature, blood pressure, blood oxygenation, pH, lactate concentration, glucose levels, or a combination thereof.

In some embodiments, the various operating parameters include confirmation of clinician training via completion of one or more on-board training modules. The display screen is further configured to display clinician messaging with the confirmation of the clinician training.

In some embodiments, only clinicians having the confirmation of the clinician training are allowed to use the system to place the medical device for vascular access.

In some embodiments, the display screen is further configured to display the clinician messaging with recommendations for one or more alternative clinicians allowed to use the system to place the medical device for vascular access in view of the one-or-more alternative clinicians having the confirmation of the clinician training.

In some embodiments, the various operating parameters include post-placement assessments as a measure of clinician proficiency for placing the medical device for vascular access. The clinician messaging with the recommendations for the one-or-more alternative clinicians allowed to use the system being further in view of the clinician proficiency.

In some embodiments, the plurality of data inputs are automatically pulled into the system by way of an electronic medical-care-facility system, which, in turn, optionally includes access to patient data via electronic medical records for patients.

In some embodiments, the electronic medical-care-facility system or the electronic medical records for the patients includes orders for specific medical devices, specific medications, or a combination thereof.

In some embodiments, the plurality of data inputs are manually input into the system by a clinician using the system. The plurality of data inputs include procedure type, one or more clinical rules, clinician experience, one or more clinician preferences, one or more orders for specific medical devices, one or more medical device-trajectory parameters, patient condition, emergent indication, difficulty in blood-vessel access, one or more orders for specific medications, one or more infusion-therapy parameters, one or more imaging parameters, dwell time, or a combination thereof.

In some embodiments, the one-or-more clinical rules include purchase length for the medical device, blood-vessel occupancy of the medical device, or both when the medical device is a catheter.

In some embodiments, the one-or-more medical device-trajectory parameters include an insertion angle, a needle-guide configuration, or both when the medical device is a needle.

In some embodiments, the one-or-more infusion-therapy parameters include fluid replacement, potassium, heparin, insulin, one or more antibiotics, one or more vesicants, one or more irritants, blood, one or more blood products, pain medication, power-injection parameters, or a combination thereof.

In some embodiments, the one-or-more imaging parameters include at least an intention for power injection.

In some embodiments, the procedure type includes at least an intention for blood aspiration.

In some embodiments, the difficulty in blood-vessel access includes a determination of whether a blood vessel is visible, palpable, torturous, valvular, or a combination thereof.

In some embodiments, the patient condition includes blood pressure, hydration, nutrition, temperature, or a combination thereof.

In some embodiments, the clinician experience includes clinician training for placing the medical device for vascular access or clinician proficiency for placing the medical device for vascular access.

Also disclosed herein is a method of a system for automatically recommending a medical device for vascular access. The method includes an instantiating step, an ultrasound image-displaying step, and medical device-recommending step. The instantiating step includes executing instructions in memory of a console by one or more processors of the console to instantiate one or more processes for the automatic recommending of the medical device for vascular access. The recommending of the medical device is in accordance with a plurality of data inputs, various operating parameters, or a combination thereof. The ultrasound image-displaying step includes displaying on a display screen optionally integrated into the console an ultrasound image obtained by an ultrasound probe operably coupled to the console. The ultrasound image includes one or more blood vessels below a skin surface of a patient. The medical device-recommending step includes displaying on the display screen the medical device recommended for vascular access of the one-or-more blood vessels in the ultrasound image.

In some embodiments, the method further includes placement-tracking step. The placement-tracking step includes tracking placement of the medical device while the medical device is being placed by a clinician in a vasculature of the patient.

In some embodiments, the method further includes a placement-confirming step. The placement-confirming step includes confirming the placement of the medical device after the medical device is placed by the clinician in the vasculature of the patient.

In some embodiments, the method further includes a placement-evaluating step. The placement-evaluating step includes evaluating the placement of the medical device after the medical device is placed by the clinician in the vasculature of the patient. The placement-evaluating step contributes to assessing clinician proficiency for placing the medical device for vascular access.

These and other features of the concepts provided herein will become more apparent to those of skill in the art in view of the accompanying drawings and following description, which describe particular embodiments of such concepts in greater detail.

### DRAWINGS

FIG. 1 illustrates a system for automatically recommending a medical device for vascular access in accordance with some embodiments.

FIG. 2 illustrates a block diagram of the system of FIG. 1 in accordance with some embodiments.

FIG. 3A illustrates a portion of a combined method including a method of a clinician using the system and a method of the system, itself, in accordance with some embodiments.

FIG. 3B illustrates another portion of the combined method including the method of the clinician using the system and the method of the system, itself, in accordance with some embodiments.

FIG. 3C illustrates yet another portion of the combined method including the method of the clinician using the system and the method of the system, itself, in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or “proximal section” of, for example, a catheter includes a portion or section of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal section, or the proximal length of the catheter can include the proximal end of the catheter; however, the proximal portion, the proximal section, or the proximal length of the catheter need not include the proximal end of the catheter. That is, unless context suggests otherwise, the proximal portion, the proximal section, or the proximal length of the catheter is not a terminal portion or terminal length of the catheter.

With respect to “distal,” a “distal portion” or a “distal section” of, for example, a catheter includes a portion or section of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal section, or the distal length of the catheter can include the distal end of the catheter; however, the distal portion, the distal section, or the distal length of the catheter need not include the distal end of the catheter. That is, unless context suggests otherwise, the distal portion, the distal section, or the distal length of the catheter is not a terminal portion or terminal length of the catheter.

With respect to “logic,” logic refers to hardware, software, or firmware configured to perform one or more functions. As hardware, logic can refer to circuitry having data-processing or storage functionality. Examples of such circuitry include, but are not limited to, a hardware processor (e.g., a microprocessor, one or more processor cores, a digital-signal processor, a programmable gate array [“PGA”], a microcontroller, an application specific integrated circuit [“ASIC”], etc.), semiconductor memory, or the like. As software, logic can refer to one or more processes, one or more instances, Application Programming Interface(s) (API), subroutine(s), function(s), applet(s), servlet(s), routine(s), source code, object code, shared or dynamic link libraries (dll), or even one or more instructions. Such software can be stored in any type of a suitable non-transitory storage medium or transitory storage medium (e.g., electrical signals, optical signals, acoustical signals, or some other form of propagated signals). Examples of a non-transitory storage medium include, but are not limited to, a programmable circuit; a non-persistent storage medium such as volatile memory (e.g., any type of random-access memory [“RAM”]); a persistent storage medium such as non-volatile memory (e.g., read-only memory [“ROM”], power-backed RAM, flash memory, phase-change memory, etc.), a solid-state drive, a hard-disk drive, an optical-disc drive, or a portable memory device. As firmware, logic can be stored in persistent storage.

As used herein a “vascular access device” can be a medical device for vascular access including, but not limited to, a catheter such as a peripherally inserted central catheter (“PICC”), a central venous catheter (“CVC”), a midline catheter, an intravenous line such as a peripheral intravenous line (“PIV”), or the like.

Unless defined otherwise, all technical and scientific terms used herein have the same meaning as commonly understood by those of ordinary skill in the art.

Again, current technologies work to optimize placement of a user-selected medical device for vascular access; however, the user-selected medical device is not always the best medical device for vascular access in view of multifaceted considerations of applicable criteria.

Disclosed herein are systems and methods for automatically recommending medical devices for vascular access to ensure best procedural outcomes starting with medical-device selection. Notably, such systems can incorporate automated data inputs, clinician-provided data inputs, or a combination thereof in decision-making processes to instantly provide clinicians recommendations for appropriate medical devices for their patients.

### Systems

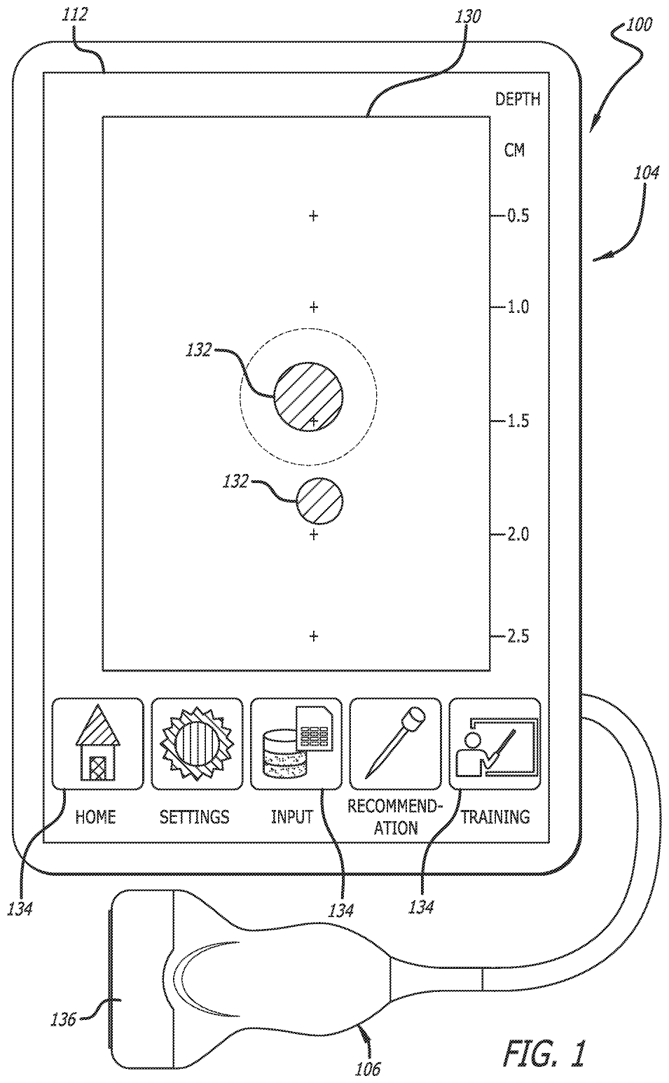

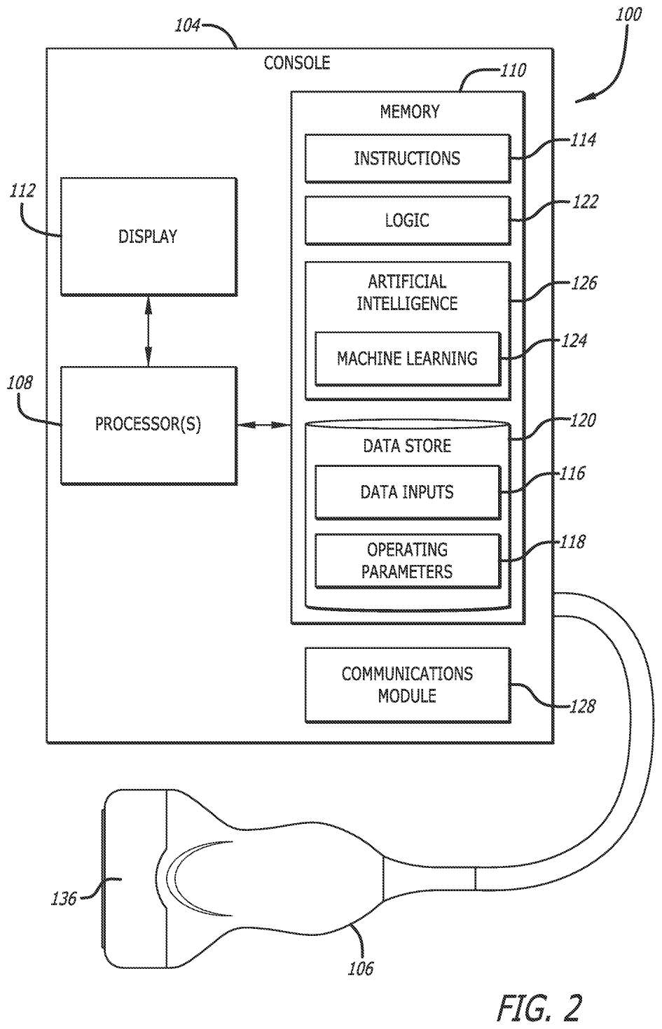

FIG. 1 illustrates a system 100 for automatically recommending a medical device 102 for vascular access in accordance with some embodiments. FIG. 2 illustrates a block diagram of the system 100 in accordance with some embodiments.

As shown, the system 100 can include a console 104 and an ultrasound probe 106 configured to operably couple with each other in a wired or wireless connection. Notably, while the system 100 is shown in FIG. 1 with the console 104, mobile devices such as smartphones or tablet computers loaded with ultrasound-imaging software can substitute for the console 104 in some embodiments.

The console 104 can include one or more processors 108 and memory 110. Optionally, the console 104 can further include a display screen 112 (e.g., touch screen).

The memory 110 can include random-access memory (“RAM”) or non-volatile memory (e.g., electrically erasable programmable read-only memory [“EEPROM”]), and the one-or-more processors 108 and the memory 110 of the console 104 can be configured to control various functions of the system 100, as well as executing various operations (e.g., processing electrical signals from the ultrasonic transducers of the ultrasound probe 106 into ultrasound images) during operation of the system 100 in accordance with executable instructions 114 therefor stored in the memory 110 for execution by the one-or-more processors 108. Indeed, the instructions 114 are configured to instantiate one or more processes when executed by the one-or-more processors 108 for the automatic recommending of the medical device 102 for vascular access. The automatic recommending of the medical device 102 for vascular access can be in accordance with a plurality of data inputs 116, various operating parameters 118, or a combination thereof stored at least temporarily (e.g. during a procedure) in a data store 120 for the automatic recommending by at least logic 122, algorithms, machine learning 124, artificial intelligence 126, or a combination thereof such as the artificial intelligence 126 (e.g., an artificial neural network) trained via the machine learning 124 using known, acceptable medical-device recommendations for the plurality of data inputs 116 or the various operating parameters 118. Though, the automatic recommending of the medical device 102 for vascular access can be further in accordance with a plurality of patient condition parameters input by an input device (e.g., thermometer, blood-pressure monitor, blood-oxygenation monitor, etc.) configured to be operably coupled to the console 104, optionally, wired or wirelessly through a communications module 128. The plurality of patient condition parameters can include temperature, blood pressure, blood oxygenation, pH, lactate concentration, glucose levels, or a combination thereof. Notably, the logic 122, the algorithms, the machine learning 124, the artificial intelligence 126, or the combination thereof can be further for automatically guiding optimum blood-vessel size, differentiating between veins and arteries, automatically detecting a needle guide, presetting an angle of insertion for the needle guide, or the like, as set forth herein.

The plurality of data inputs 116 can be automatically input into the system 100 or manually input into the system 100 by a clinician using the system 100.

As to automatic input of the plurality of data inputs 116 into the system 100, the plurality of data inputs 116 can be automatically determined by the system 100 by way of the logic 122, the algorithms, the machine learning 124, the artificial intelligence 126, or the combination thereof such as from ultrasound imaging with the system 100. For example, the plurality of data inputs 116 can include blood-vessel size, blood-vessel location, blood-vessel cross section, blood-vessel thickness, or difficulty in blood-vessel access, optionally, based upon a vascular wellness assessment using at least some of the foregoing data inputs 116. The blood-vessel location can include a determination of a distance from an acoustic lens of the ultrasound probe 106 to a top of a blood vessel, a bottom of the blood vessel, or both. In addition, the blood-vessel location can include a determination of a distance from one blood vessel to another blood vessel. A largest distance between the acoustic lens of the ultrasound probe 106 and the bottom of the blood vessel can be a determination parameter. The vascular wellness assessment can include a determination of whether a blood vessel is visible, palpable, torturous, valvular, or a combination thereof.

Further as to the automatic input of the plurality of data inputs 116 into the system 100, the plurality of data inputs 116 can be automatically pulled into the system 100 by way of the communications module 128 for communicating with an electronic medical-care-facility system, which, in turn, optionally includes access to patient data via electronic medical records for patients. The electronic medical-care-facility system 100 or the electronic medical records for the patients can include orders for specific medical devices, specific medications (e.g., infusates), or a combination thereof for use by the logic 122, the algorithms, the machine learning 124, the artificial intelligence 126, or the combination thereof for the automatic recommending of the medical device 102 for vascular access. In addition, the electronic medical-care-facility system 100 or the electronic medical records for the patients can include additional patient data from, for example, diagnostics or imaging such as digital intravenous angiography (“DIVA”) for use by the logic 122, the algorithms, the machine learning 124, the artificial intelligence 126, or the combination thereof for the automatic recommending of the medical device 102 for vascular access.

As to manual input of the plurality of data inputs 116 into the system 100, the plurality of data inputs 116 can be manually input into the system 100 by a clinician using the system 100. For example, the plurality of data inputs 116 can include procedure type, one or more clinical rules, optionally, preloaded based upon common clinical standards, clinician experience, one or more clinician preferences, one or more orders for specific medical devices, one or more medical device-trajectory parameters, patient condition, emergent indication, difficulty in blood-vessel access, one or more orders for specific medications, one or more infusion-therapy parameters, one or more imaging parameters, dwell time, or a combination thereof. The procedure type can include at least an intention for blood aspiration. The one-or-more clinical rules can include purchase length for the medical device 102, blood-vessel occupancy of the medical device 102, or both when the medical device 102 is a catheter. The clinician experience can include clinician training, clinician proficiency, or both for placing the medical device 102 for vascular access. The one-or-more medical device-trajectory parameters can include an insertion angle (e.g., an assumed insertion angle), a needle-guide configuration (e.g., an assumed needle-guide configuration), or both when the medical device 102 is a needle. The patient condition can include blood pressure, hydration, nutrition, temperature, or a combination thereof. The difficulty in blood-vessel access can include a determination of whether a blood vessel is visible, palpable, torturous, valvular, or a combination thereof. The one-or-more infusion-therapy parameters can include fluid replacement, potassium, heparin, insulin, one or more antibiotics, one or more vesicants, one or more irritants, blood, one or more blood products, pain medication, power-injection parameters, or a combination thereof. The one-or-more imaging parameters can include at least an intention for power injection.

The various operating parameters 118, like the plurality of data inputs 116 set forth above, can be automatically input into the system 100 or manually input into the system 100 by the clinician using the system 100. The various operating parameters 118 can include confirmation of clinician training via completion of one or more on-board training modules. Clinicians having the confirmation of the clinician training can be allowed to use the system 100 to place the medical device 102 for vascular access, whereas those not having the confirmation of the clinician training can be restricted from using the system 100 to place the medical device 102 for vascular access. The various operating parameters 118 can further include post-placement assessments as a measure of clinician proficiency for placing the medical device 102 for vascular access. As set forth below, the display screen 112 can be configured to display the clinician messaging. Such clinician messaging can include the confirmation of the clinician training or recommendations for one or more alternative clinicians allowed to use the system 100 to place the medical device 102 for vascular access in view of the one-or-more alternative clinicians having the confirmation of the clinician training. Notably, the clinician messaging can include the recommendations for the one-or-more alternative clinicians allowed to use the system 100, which can be further in view of the clinician proficiency.

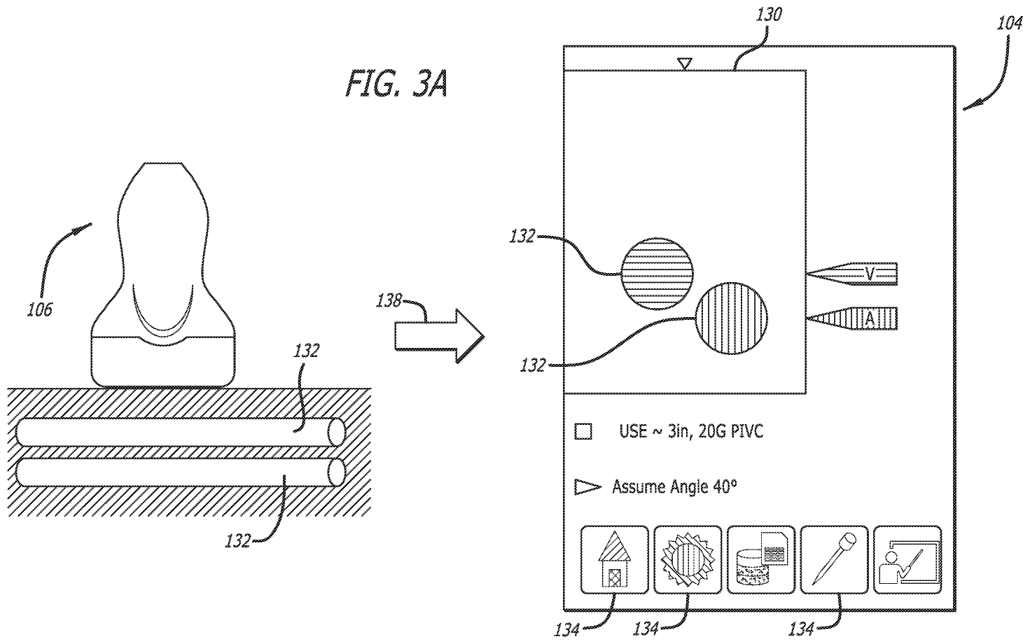

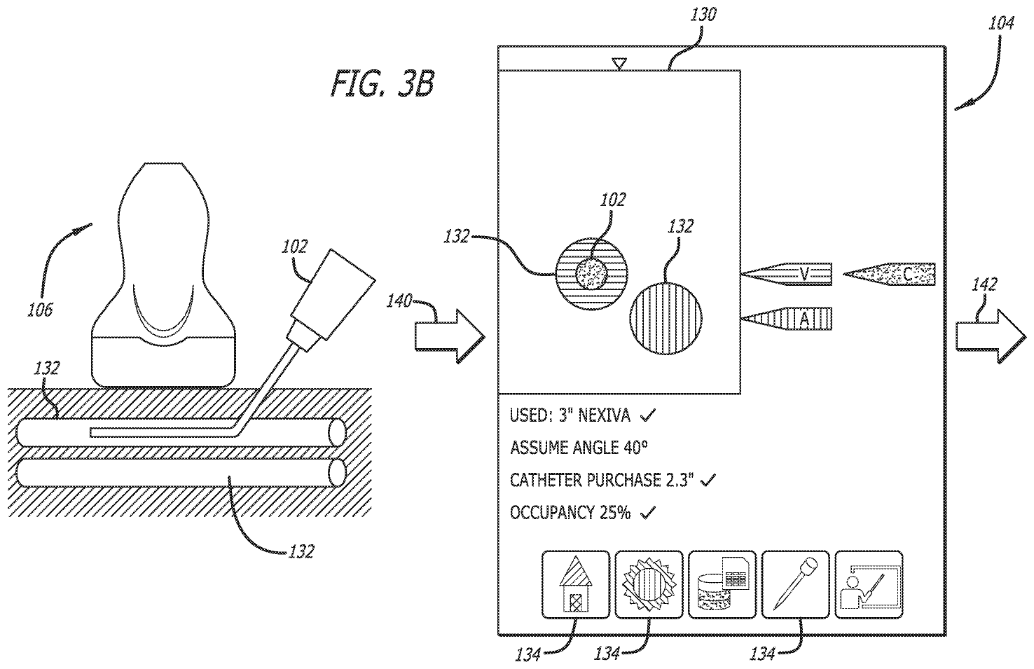

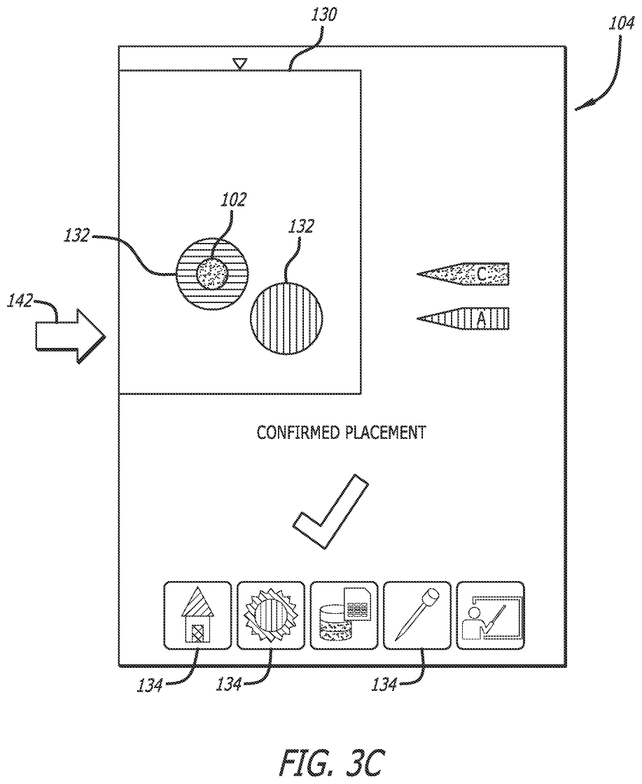

The display screen 112 can be integrated into the console 104, as shown, or the display screen 112 can be part of a standalone monitor configured to operably couple with the console 104. The display screen 112 can be configured to display an ultrasound image 130 including one or more blood vessels 132 below a skin surface of a patient, for example, as shown in FIGS. 3A and 3B by an on-screen indicator labeled “V” for a vein and an on-screen indictor labeled “A” for an artery. The display screen 112 can also be configured to display the medical device 102 recommended for vascular access of the one-or-more blood vessels 132 in the ultrasound image 130. Notably, the display screen 112 can also be configured to display one or more on-screen buttons 134 (e.g., a home button, a settings button, a data-input button, a medical-device recommendation button, a training button, etc.) enabling the clinician to interact with various aspects of the system 100. For example, the one-or-more on-screen buttons 134 can include the example medical-device recommendation button, which the clinician can press when any preassessment of the patient is complete for placing the medical device 102. (See FIG. 3A for the preassessment of the patient.) In addition, the display screen 112 can be further configured to display clinician messaging such as the confirmation of the clinician training or the one-or-more alternative clinicians allowed to use the system 100 set forth above.

While not shown, the console 104 can further include a power connection configured to enable an operable connection to an external power supply. An internal power supply (e.g., a battery) can also be employed either with or exclusive of the external power supply. Power management circuitry of the console 104 can regulate power use and distribution.

The ultrasound probe 106 can include a probe head 136 housing an array of ultrasonic transducers, wherein the ultrasonic transducers are piezoelectric ultrasonic transducers or capacitive micromachined ultrasonic transducers (“CMUTs”). The probe head 136 is configured for placement against the skin surface of the patient proximate a prospective site for placing the medical device 102 for vascular access, where the ultrasonic transducers in the probe head 136 can generate ultrasound signals and emit the generated ultrasound signals into the patient in a number of pulses, receive reflected ultrasound signals or ultrasound echoes from the patient by way of reflection of the generated ultrasonic pulses by the body of the patient, and convert the reflected ultrasound signals into corresponding electrical signals for processing into the ultrasound image 130 by the console 104.

Advantageously, the system 100 can be used or otherwise incorporated into a robotic system for automatically placing recommended medical devices for vascular access.

### Methods

FIGS. 3A-3C illustrate a combined method including a method of a clinician using the system 100 and a method of the system 100, itself, in accordance with some embodiments.

Methods can include a method of the system 100 for automatically recommending a medical device (i.e., the medical device 102 set forth above) for vascular access. Such a method can include one or more steps selected from an instantiating step, an ultrasound image-displaying step, a medical device-recommending step, a placement-tracking step, a placement-confirming step, and a placement-evaluating step.

The instantiating step can include executing the instructions 114 in the memory 110 of the console 104 by the one-or-more processors 108 of the console 104 to instantiate the one-or-more processes for the automatic recommending of the medical device 102 for vascular access. The instantiating step can be initiated by a clinician by simply powering up the system 100.

The ultrasound image-displaying step, which is indicated as step 138 in FIG. 3A, can include displaying on the display screen 112 optionally integrated into the console 104 an ultrasound image (i.e., the ultrasound image 103 set forth above) obtained by the ultrasound probe 106 operably coupled to the console 104. The ultrasound image 130 can include one or more blood vessels (i.e., the one-or-more blood vessels 132 set forth above) below a skin surface of a patient, which is useful to the clinician during preassessment of the patient for placing the medical device 102. Again, as shown in FIG. 3A, the on-screen indicator labeled “V” can be used to indicate a vein and the on-screen indictor labeled “A” can be used to indicate an artery.

The medical device-recommending step, which is also shown in the step 138, can include displaying on the display screen 112 the medical device 102 recommended for vascular access of the one-or-more blood vessels 132 in the ultrasound image 130. As set forth above, the recommending of the medical device 102 can be in accordance with the plurality of data inputs 116, the various operating parameters 118, or the combination thereof. Optionally, the displaying on the display screen 112 of the medical device 102 recommended for vascular access can be subsequent to the clinician requesting a recommendation for the medical device 102 via the one-or-more on-screen buttons 134 such as the medical-device recommendation button.

The placement-tracking step, which is shown as step 140 in FIG. 3B, can include tracking placement of the medical device 102 while the medical device 102 is being placed by the clinician in a vasculature of the patient. As shown in FIG. 3B, an on-screen indicator labeled “C” can be used to indicate, for example, a catheter as the medical device 102.

The placement-confirming step, which is shown as step 142 in FIG. 3C, can include confirming the placement of the medical device 102 after the medical device 102 is placed by the clinician in the vasculature of the patient.

While not shown in FIG. 3C, the placement-evaluating step can include evaluating the placement of the medical device 102 after the medical device 102 is placed by the clinician in the vasculature of the patient. The placement-evaluating step can contribute to assessing clinician proficiency for placing the medical device 102 for vascular access.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures can be made from the particular embodiments disclosed herein without departing from the scope of the concepts provided herein.

What is claimed is:

1. A system for automatically recommending a medical device for vascular access, comprising:
an ultrasound probe;
a console operably coupled to the ultrasound probe, the console including:
one or more processors;
memory including instructions configured to instantiate one or more processes when executed by the one-or-more processors for the automatic recommending of the medical device for vascular access in accordance with a plurality of data inputs, various operating parameters, or a combination thereof, the automatic recommending of the medical device using at least logic, algorithms, machine learning, artificial intelligence artificial intelligence, or a combination thereof; and
a display screen optionally integrated into the console, the display screen configured to display:
an ultrasound image including one or more blood vessels below a skin surface of a patient; and
the medical device recommended for vascular access of the one-or-more blood vessels in the ultrasound image.

2. The system of claim 1, wherein the plurality of data inputs are automatically determined by the system by way of the logic, the algorithms, the machine learning, the artificial intelligence, or the combination thereof.

3. The system of claim 2, wherein the plurality of data inputs are selected from blood-vessel size, blood-vessel location, blood-vessel cross section, blood-vessel thickness, and difficulty in blood-vessel access.

4. The system of claim 3, wherein the blood-vessel location includes a determination of a distance from a lens of the ultrasound probe to a top of a blood vessel, a bottom of the blood vessel, or both.

5. The system of claim 4, wherein a largest distance between the lens of the ultrasound probe and the bottom of the blood vessel is a determination parameter.

6. The system of claim 3, wherein the difficulty in blood-vessel access includes a determination of whether a blood vessel is visible, palpable, torturous, valvular, or a combination thereof.

7. The system of claim 2, wherein the logic, the algorithms, the machine learning, the artificial intelligence, or the combination thereof are further for automatically guiding optimum blood-vessel size, differentiating between veins and arteries, or both.

8. The system of claim 2, wherein the logic, the algorithms, the machine learning, the artificial intelligence, or the combination thereof are further for automatically detecting a needle guide and presetting an angle of insertion for the needle guide.

9. The system of claim 1, wherein the automatic recommending of the medical device for vascular access is further in accordance with a plurality of patient condition parameters input by an input device, the plurality of patient condition parameters including temperature, blood pressure, blood oxygenation, pH, lactate concentration, glucose levels, or a combination thereof.

10. The system of claim 1, wherein the various operating parameters include confirmation of clinician training via completion of one or more on-board training modules, the display screen further configured to display clinician messaging with the confirmation of the clinician training.

11. The system of claim 10, wherein only clinicians having the confirmation of the clinician training are allowed to use the system to place the medical device for vascular access.

12. The system of claim 11, wherein the display screen is further configured to display the clinician messaging with recommendations for one or more alternative clinicians allowed to use the system to place the medical device for vascular access in view of the one-or-more alternative clinicians having the confirmation of the clinician training.

13. The system of claim 12, wherein the various operating parameters include post-placement assessments as a measure of clinician proficiency for placing the medical device for vascular access, the clinician messaging with the recommendations for the one-or-more alternative clinicians allowed to use the system being further in view of the clinician proficiency.

14. The system of claim 1, wherein the plurality of data inputs are automatically pulled into the system by way of an electronic medical-care-facility system, which, in turn, optionally includes access to patient data via electronic medical records for patients.

15. The system of claim 1, wherein the electronic medical-care-facility system or the electronic medical records for the patients includes orders for specific medical devices, specific medications, or a combination thereof.

16. The system of claim 1, wherein the plurality of data inputs are manually input into the system by a clinician using the system, the plurality of data inputs including procedure type, one or more clinical rules, clinician experience, one or more clinician preferences, one or more orders for specific medical devices, one or more medical device-trajectory parameters, patient condition, emergent indication, difficulty in blood-vessel access, one or more orders for specific medications, one or more infusion-therapy parameters, one or more imaging parameters, dwell time, or a combination thereof.

17. The system of claim 16, wherein the one-or-more clinical rules include purchase length for the medical device, blood-vessel occupancy of the medical device, or both when the medical device is a catheter.

18. The system of claim 16, wherein the one-or-more medical device-trajectory parameters include an insertion angle, a needle-guide configuration, or both when the medical device is a needle.

19. The system of claim 16, wherein the one-or-more infusion-therapy parameters include fluid replacement, potassium, heparin, insulin, one or more antibiotics, one or more vesicants, one or more irritants, blood, one or more blood products, pain medication, power-injection parameters, or a combination thereof.

20. The system of claim 16, wherein the one-or-more imaging parameters include at least an intention for power injection.

21. The system of claim 16, wherein the procedure type includes at least an intention for blood aspiration.

22. The system of claim 16, wherein the difficulty in blood-vessel access includes a determination of whether a blood vessel is visible, palpable, torturous, valvular, or a combination thereof.

23. The system of claim 16, wherein the patient condition includes blood pressure, hydration, nutrition, temperature, or a combination thereof.

24. The system of claim 16, wherein the clinician experience includes clinician training for placing the medical device for vascular access or clinician proficiency for placing the medical device for vascular access.

25. A method of a system for automatically recommending a medical device for vascular access, comprising:
executing instructions in memory of a console by one or more processors of the console to instantiate one or more processes for the automatic recommending of the medical device for vascular access in accordance with a plurality of data inputs, various operating parameters, or a combination thereof; and
displaying on a display screen optionally integrated into the console an ultrasound image obtained by an ultrasound probe operably coupled to the console, the ultrasound image including one or more blood vessels below a skin surface of a patient; and
displaying on the display screen the medical device recommended for vascular access of the one-or-more blood vessels in the ultrasound image.

26. The method of claim 25, further comprising tracking placement of the medical device while the medical device is being placed by a clinician in a vasculature of the patient.

27. The method of claim 26, further comprising confirming the placement of the medical device after the medical device is placed by the clinician in the vasculature of the patient.

28. The method of claim 27, further comprising evaluating the placement of the medical device after the medical device is placed by the clinician in the vasculature of the patient, the evaluating of the placement of the medical device contributing to assessing clinician proficiency for placing the medical device for vascular access.
